(19) United States
(12) Patent Application Publication
(10) Pub. No.: US 2008/0003184 A1
(43) Pub. Date: Jan. 3, 2008

(54) Superparamagnetic Gadolinium Oxide Nanoscale Particles and Compositions Comprising Such Particles

(76) Inventors: Kajsa Uvdal, Linkoping (SE); Maria Engstrom, Linkoping (SE)

Correspondence Address: Dinsmore & Shohl, 1900 Chemed Center, 255 East Fifth Street, Cincinnati, OH, 45202, US

(21) Appl. No.: 11/662,674

(22) PCT Filed: Sep. 14, 2005
(86) PCT No.: PCT/SE05/01335

§ 371 Date: Mar. 13, 2007

Related U.S. Application Data
(60) Provisional application No. 60/609,740, filed on Sep. 14, 2004.
(60) Provisional application No. 60/682,078, filed on May 18, 2005.

(51) Int. Cl. A61K 49/18; A61B 5/055

(52) U.S. Cl. 424/9.323; 424/9.32

(57) ABSTRACT

Superparamagnetic nanoscale particles are disclosed which are useful for providing a contrast agent with high signal intensity, high relaxivity and high intrinsic magnetism. The disclosed contrast agents will have utility and magnetic resonance imaging (MRI) and associated techniques.

### FIELD OF INVENTION

The present invention relates to superparamagnetic gadolinium oxide nanoparticles and their utility in selective tissue imaging as well as cell or molecular analysis.

### BACKGROUND OF INVENTION

High spatial resolution and the unique ability to distinguish soft tissue have made magnetic resonance imaging (MRI) one of the most important tools for medical image diagnostics. The presence of MRI contrast agents influence the image by altering the relaxation times T₁ and T₂ of hydrogen nuclei. Different hydrogen relaxation times in different tissues cause image contrast in MRI. There are two types of hydrogen relaxation times in MRI, T₁ and T₂. T₁ is called longitudinal relaxation time and determines the return of the magnetisation to equilibrium after a perturbation by a magnetic field pulse. T₂ is called transversal relaxation time and determines the dephasing of the signal due to interaction between magnetic moments. In addition, T₂* (“T₂ star”) is the actual transversal relaxation time that also includes effects by magnetic field inhomogeneities. The effect of reducing T₁ is signal increase and the effect of reducing T₂ is signal decrease. Contrast agents can be classified either as positive or negative agents depending on whether the signal is increased or decreased in the presence of the contrast media.

All contrast agents influence both relaxation times, but some agents have predominant effect on either T₁ or T₂. Several properties of the paramagnetic element of the contrast agent influence the contrast of MR images. The most important properties are the magnetic moment, the electron relaxation time, and the ability to co-ordinate water either in the inner or outer co-ordination sphere. Rotation of the paramagnetic agent, diffusion, and water-exchange are also important mechanisms. The signal from a spin echo sequence as a function of scanning parameters can be expressed as:
S(TR,TE)=ρe^{-TE/T2}(1−e^{-TR/T1}) (1)
where ρ=spin density, TE=echo time, and TR=repetition time. From Eq. 1 it can be seen that the relaxation times influences the signal to a high extent. There is a competitive relation between the two relaxation times, which explains the peak in the signal versus contrast agent concentration that has been observed. The relaxation rate, (1/Tᵢ, i=1, 2) observed is proportional to the concentration (C) of the contrast agent:
1/Tᵢ(observed)=1/Tᵢ(inherent)+rᵢC (2)
where 1/Tᵢ (observed) is the relaxation time in presence of the contrast agent, 1/Tᵢ (inherent) is the inherent tissue relaxation time, and rᵢ is the relaxivity constant.

Due to their magnetic properties, ionic complexes (chelates) of Gd³⁺ are commonly used as contrast agents in clinical MRI. However, the weak signal intensity enhancement of such agents is insufficient for molecular imaging. With growing desire for better contrast, better delineation of different tissues, there is an increasing demand for contrast agents with greater signal intensity enhancement. Selective imaging of atherosclerotic plaques or pulmonary emboli are examples of novel MRI applications with a huge potential for early diagnosis of widespread diseases. In a new generation of MRI contrast media, biocompatible nanoparticles with unique magnetic properties are highly interesting for development. Superparamagnetic nanoparticles have advantageous properties for molecular imaging compared to chelates as they have higher relaxivity per molecular binding site. Thus, new methods for magnetic tracing by superparamagnetic nanoparticles provide new possibilities for in vivo cell and molecular MRI, see Jaffer F A et al., JAMA, 2005; 293: 855-862; Gillies R J. J Cell Biochem. 2002; 39: 231-238; Dijkhuizen R M, et al. J Cerebral Blood Flow and Metabolism, 2003; 23: 1383-1402; and Wickline S A et al., J Cellular Biochemistry. 2002; S 39: 90-97. Superparamagnetic iron oxide (SPIO) particles have been explored for novel clinical applications and molecular imaging (Perez et al. Nature Biotech 20:816 (2002)). SPIOs have a very high T₂ relaxation effect, which makes them suitable for T₂-mapping of cell and molecular interactions. However SPIOs cause signal loss due to susceptibility artefacts. These artefacts are shown in the image as signal voids that cannot be distinguished from tissue voids. Such artefacts can also impede delineation of fine structures in the tissue. These are the major disadvantages of negative contrast agents.

US Patent Application 2004/0156784 (Haase et al.) describes particles made from gadolinium phosphate, which demonstrate a 100-200% improved signal intensity compared to water. However, there is no capping method suggested for particle size control, so it appears likely that there will be difficulties to obtain sufficiently small particle sizes of 1-10 nm with this method, as is needed for superparamagnetic properties.

Another type of particles comprising gadolinium is discussed by Morawski et al. in Magnetic Resonance in Medicine, 51:480 (2004), wherein it is suggested to quantify molecular epitopes in picomolar concentration in single cells with clinical MRI equipment using perfluorocarbon nanoparticles loaded with gadolinium ( )). These particles have, however, a relatively large size (˜250 nm) and do not exhibit superparamagnetism.

Magnetic particle imaging (MPI) has newly been presented as a technique for high-resolution imaging. This technique applies directly to the magnetic properties of the contrast agent itself and not to the indirect influence on proton relaxation times, which is the mechanism of conventional contrast agents. MPI has a potential for both high spatial resolution and high sensitivity. The proof of principle of MPI (Nature, June 2005) is shown, though the practical use is not yet explored. Future MPI will rely on detection of magnetic particles with strong intrinsic magnetism and superparamagnetism would be a desirable characteristic.

For reasons outlined above, there is a need of contrast agent with high signal intensity, with a high relaxivity and with high intrinsic magnetism. The present invention provides biocompatible, supeparamagnetic rare earth nanoparticles which can be used as contrast agents to meet the mentioned requirements.

### DESCRIPTION OF THE INVENTION

It is an object of the present invention to provide superparamagnetic particles, which admit an excellent contrast enhancement when used in compositions with low concentrations of active material, in a magnetic resonance imaging application.

It is also an object of the present invention to improve contrast properties of a contrast agent so that molecular imaging or imaging of cellular process is admitted.

It is another object of the present invention to provide biocompatible nanoparticles suitable for labelling with tissue specific ligands in order to enable contrast agent accumulation at a desired tissue.

The present invention as described in the following section aims at providing a gadolinium based nanoparticulate formulation which meets the mentioned requirements. Generally, the present invention relates to superparamagnetic nanoscale particles comprising a rare earth metal oxide having average sizes below 50 nm, preferably from about 0.1 to 50 nm, and more preferably from about 1 to 15 nm. Such particles may typically include one or several fractions of particles within the mentioned size ranges. Preferred rare earth metal oxides include oxides of gadolinium and dysprosium. Especially preferred is particles comprising gadolinium oxide, in particular Gd₂O₃. The inventive mentioned particles may further comprise small fractions of additional materials such as ferrous materials in order to modify their characteristics. A synthesis method of gadolinum oxide nanoparticles, described in the following experimental section, yields particles in a size between about 0.5-15 nm with a medium size of about 4 nm. By means of size fractionation, fractions with narrow distribution: 1-3 nm, 3-6 nm, 6-9 nm, 9-15 nm are obtainable.

Superparamagnetism occurs when the material is composed of very small crystallite structures (approximately 1-15 nm). The dipoles of the material have the same direction and the resulting magnetic moment of the entire crystallite will align with an external magnetic field. In this case, even though the temperature is below Curie or Néel temperature and the thermal energy is too low to overcome the coupling forces between neighboring atoms, the thermal energy is sufficiently high to change the direction of magnetization of the entire crystallite. Most importantly, the nanoscale particles according to the present invention, and compositions including the particles, exhibit superparamagnetic properties. The particles of the present invention preferably have a biocompatible and/or biospecific coating. The introduction of a coating is generally a part of the particle production process and several such processes are demonstrated in the following experimental part of the application. The coatings serve to counteract agglomeration of the particles to larger units and consequential loss of superparamagnetism; to render the particles compatible in a selected biological environment; and/or to enable the introduction of a certain biospecificity. Suitable coatings, include, but are not limited to diethylene glycol (DEG), polyethylene glycol, citric acid, oleic acid, 16-hydroxyhexadecanoic acid, 16-aminohexadecanoic acid, hexadecylamine, or trioctylphosphine oxide (TOPO). More preferably the coatings comprise diethylene glycol (DEG) and/or citric acid. In a preferred embodiment the particle have an average size of about 5 nm and have a coating comprising diethylene glycol (DEG). In accordance with another specific embodiment, the coating comprises polyethylene glycol linked to folic acid, for example with an amide bond or a spacing group, thereby providing particles with increased specificity for tumour tissues. The present invention also relates to compositions including the mentioned superparamagnetic particles. The compositions will have typical utility as contrast agents for magnetic resonance imaging (MRI). The compositions will include suitable adjuvants or excipients, including, but not limited to, pH adjusters, isotonicity adjusters and/or other agents suitable for administration to the whole body, a specific body site or a tissue sample, for example by parenteral administration. Suitably, the concentration of gadolinium in such composition will be in the range of 0.01 to 500 mM, preferably between about 0.01 to 5 mM and more preferably between about 0.01 to 2.5 mM. The concentration of the agent to be administered largely depends on the dose desired and needed for a specific application, so for this reason broad ranges are given. However, it is expected that concentrations and provided doses can be significantly reduced with the present invention.

A composition comprising the inventive gadolinium oxide based superparamagnetic particles has a capacity to reduce relaxation times T₁ and/or T₂ of neighboring hydrogen nuclei in a proton rich environment below values of T₁ and/or T₂ obtainable by a composition of a ionic complex of gadolinium. Further, a contrast agent based on the mentioned compositions will have at least 500%, preferably more than 700% greater signal intensity than water and will provide higher signal intensity than obtained by nanoscale iron oxide particles in the concentration range of 0.1 mM to 1.5 mM. This comparison is performed over the same concentration range (mol of metal atom) with comparable metal particle sizes using a commercially available iron based preparation as a reference, as will be explained in more detail below.

The following exemplifying description of the invention shows that the invented nanosized particles and compositions including such particles can provide high contrast enhancement and significantly improved relaxivity compared to state of the art, ion complexes. Accordingly, the inventive nanoparticles and compositions thereof can find utility for cell tracking with high differentiation with respect to gadolinium concentration and will find use in methods of performing MRI (magnetic resonance imaging) for studying molecular interactions or cellular processes. In addition, the presently invented superparamagnetic particles and compositions including them will admit development of methodologies for studying plaques in blood vessels in order to support an early diagnosis of arteriosclerosis, diagnosis of embolisms, tracking of implanted cells, as well as the early onset mechanisms of other pathologic conditions which so far are difficult or impossible to diagnose and treat until widespread damages are a fact. In particular, it is envisioned that the present invention will be useful to discern early stage pathologic conditions and survey the development of an elected therapy as an adjunct tool for determining the therapeutic efficacy. This would improve the possibilities to optimize doses of administrated therapeutic agents, and to provide to an early indication of the need to replace or supplement the elected therapy.

### DETAILED AND EXEMPLIFYING DESCRIPTION OF INVENTION

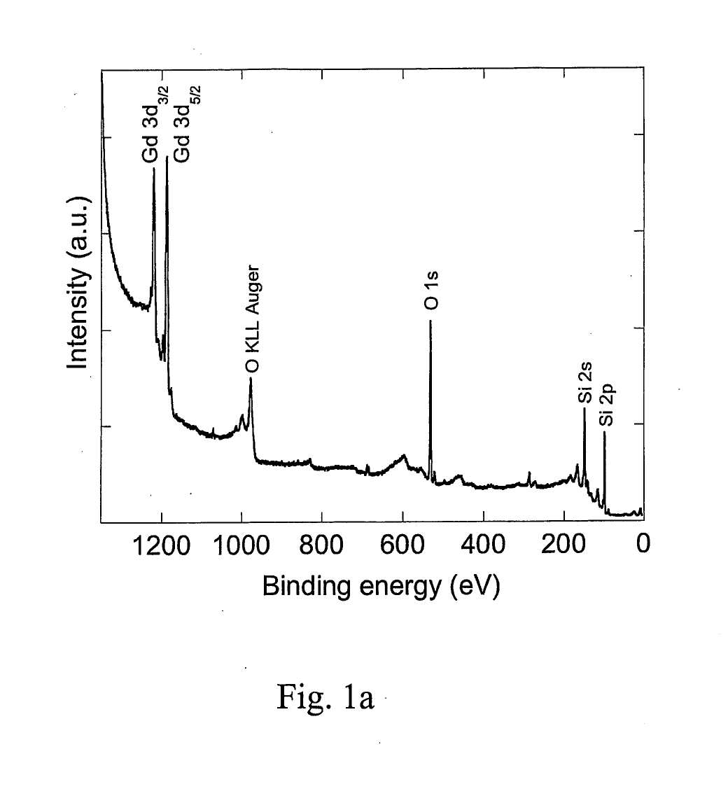

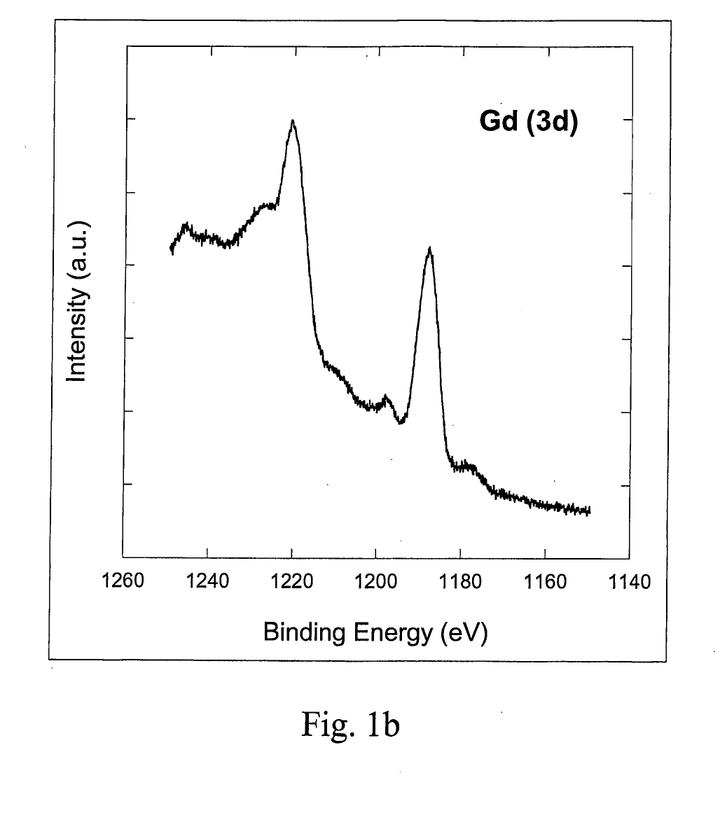

FIG. 1a to 1f show wide scan XPS spectra of different synthesised Gd₂O₃ nanoparticles spin-coated onto a silicon substrate.

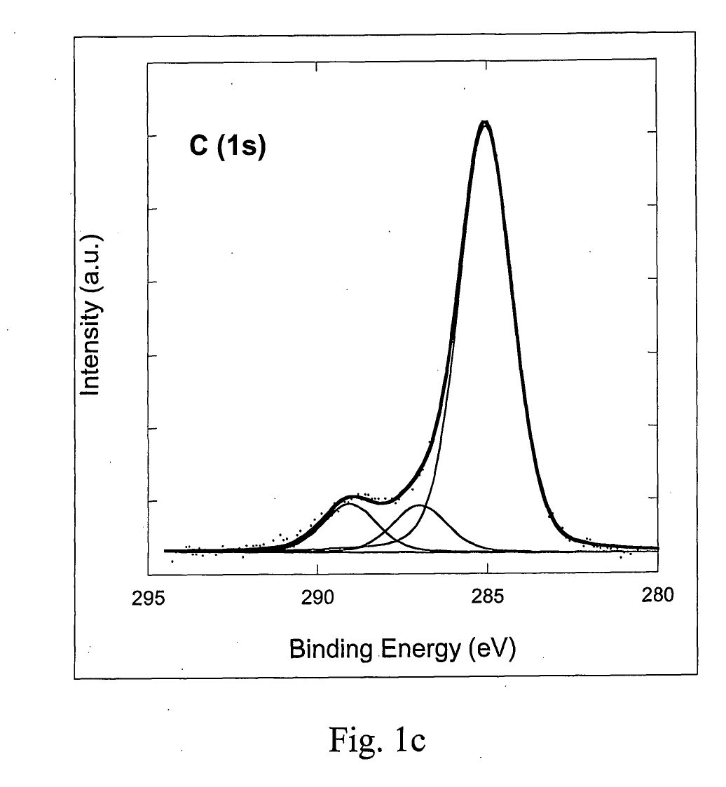

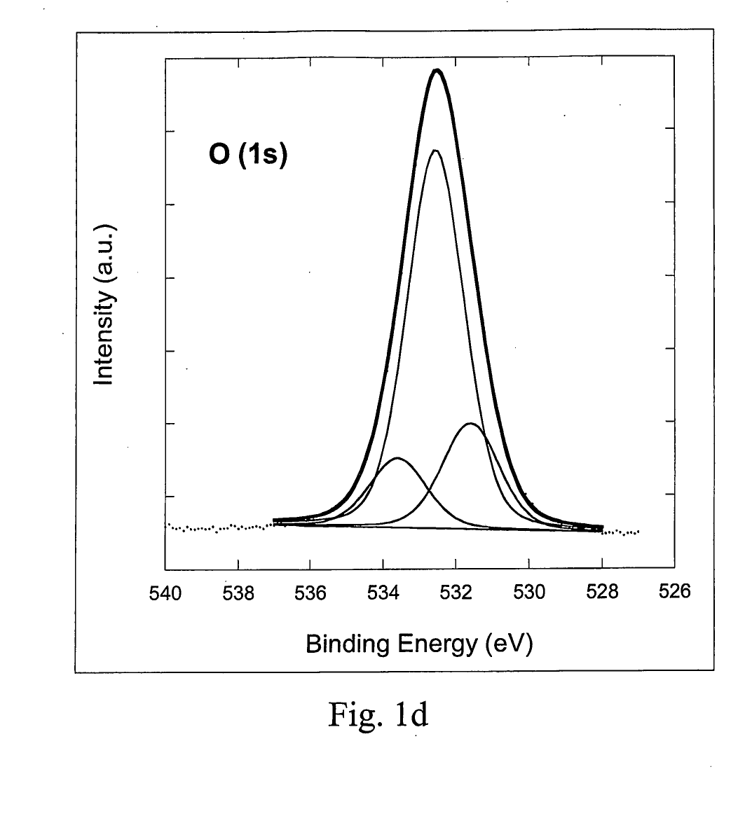

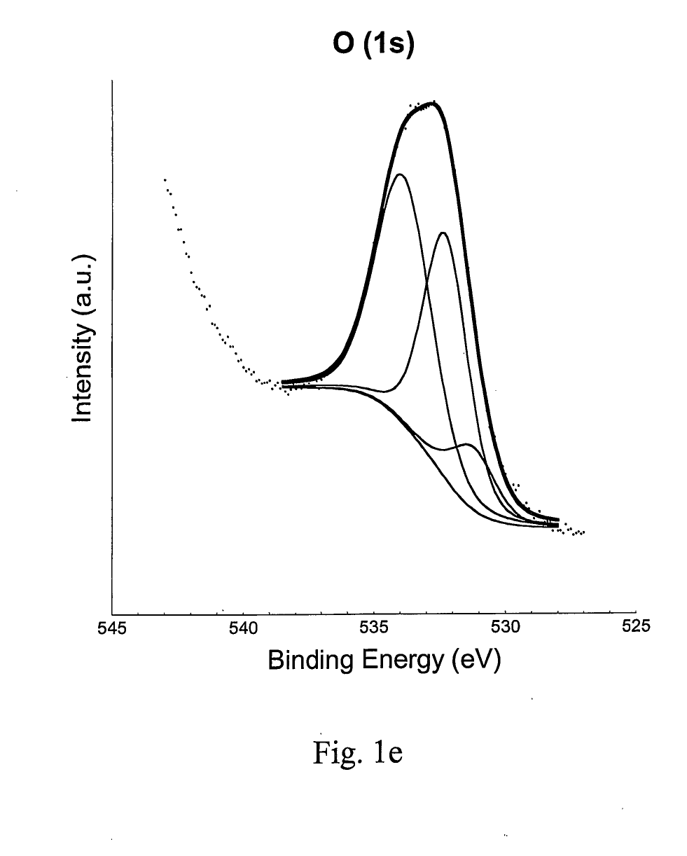

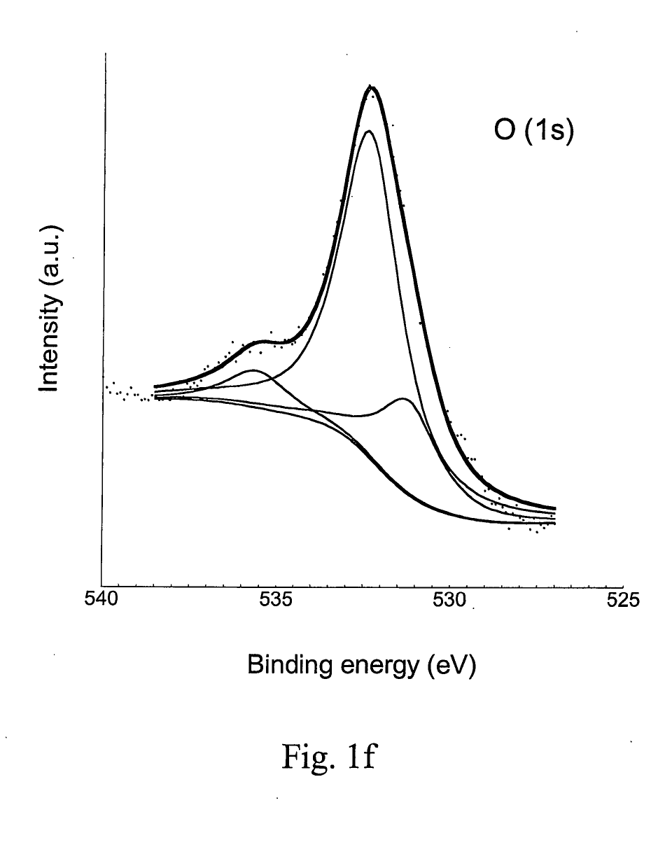

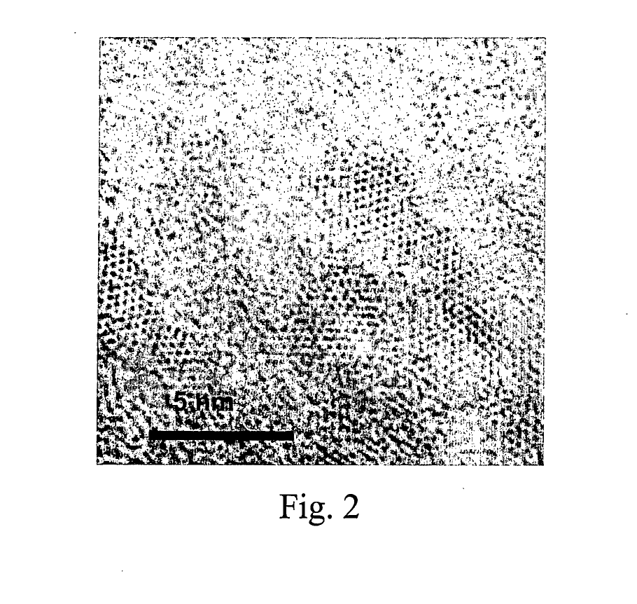

FIG. 2 is a HREM micrograph of Gd₂O₃ nanocrystals capped with DEG.

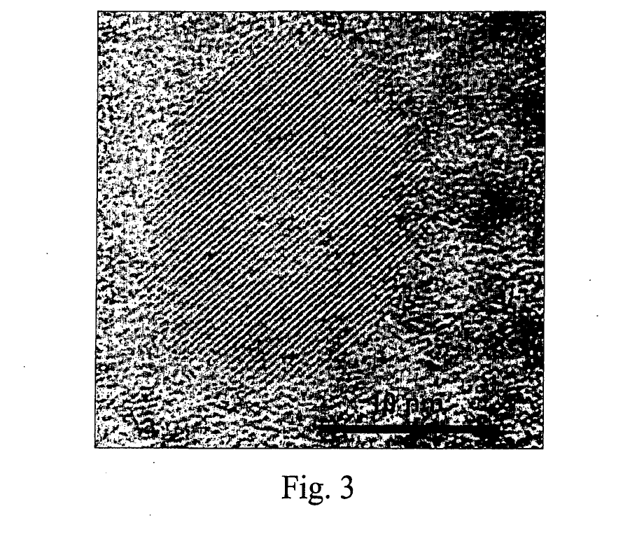

FIG. 3 is a HREM micrograph of Gd₂O₃ nanocrystals capped with oleic acid with the (222) planes visible.

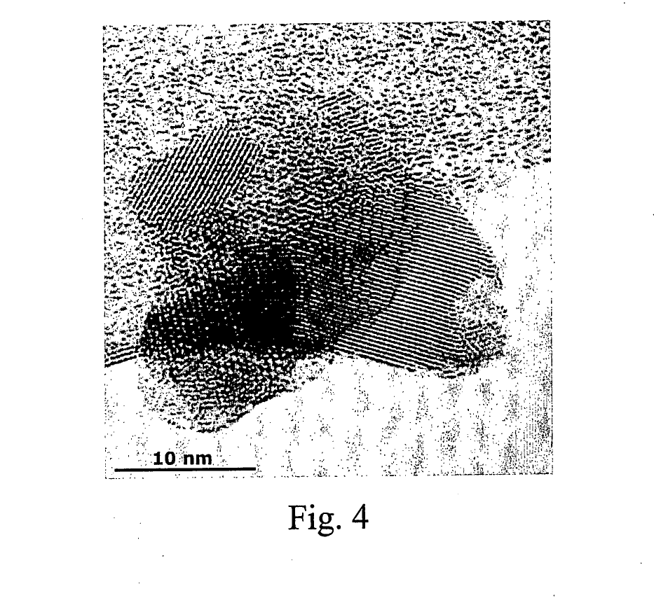

FIG. 4 is a HREM micrograph of Gd₂O₃ nanocrystals from the combustion synthesis.

FIGS. 5a and 5b show relaxivity in the form of plots of 1/Tᵢ vs. gadolinium concentration for Gd₂O₃ nanoparticles according to the present invention and Gd-DTPA (Magnevist)

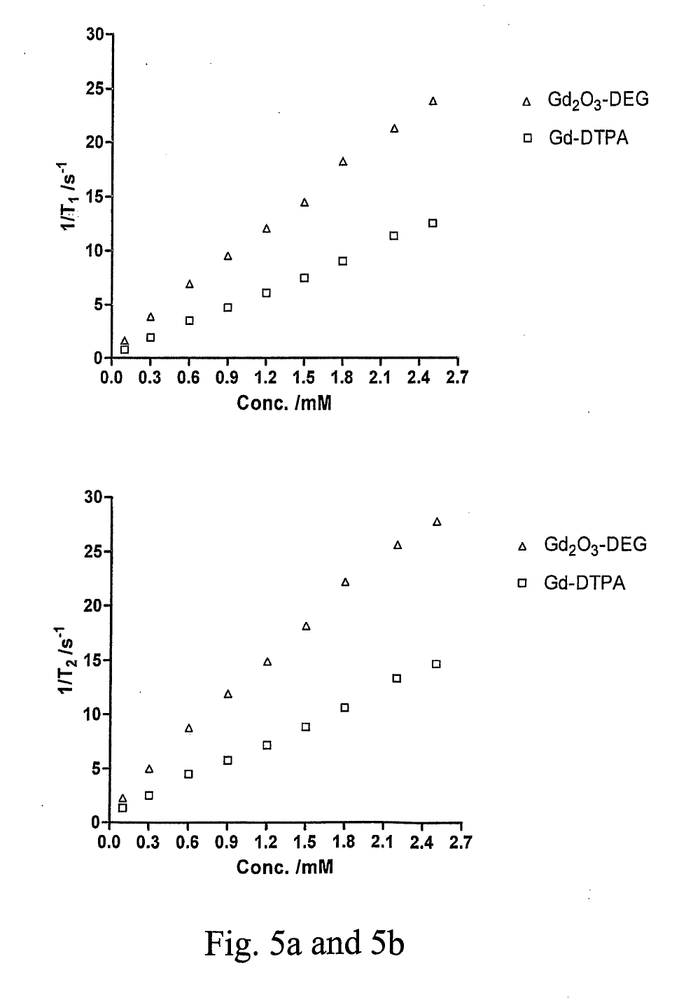

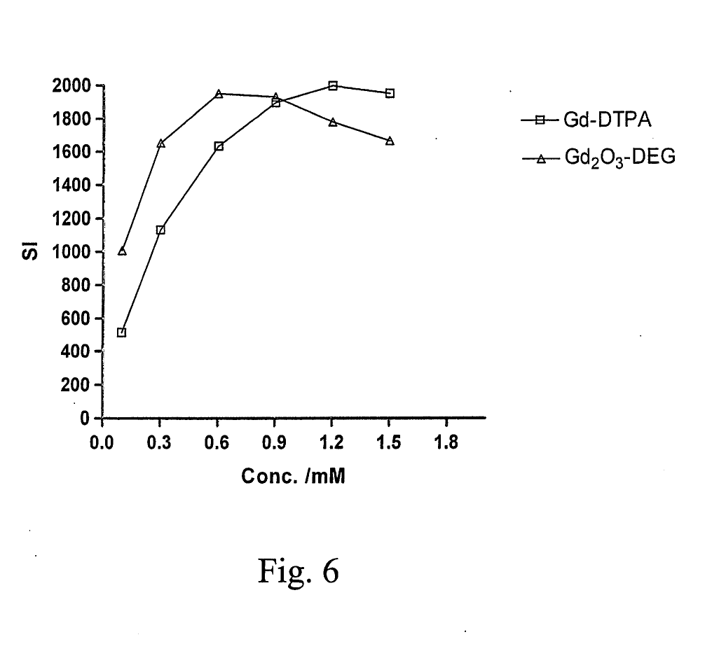

FIG. 6 shows signal intensity from first echo (TE=30 ms, TR=500 ms) in the spin echo sequence used for relaxation time measurements of FIGS. 5a and 5b.

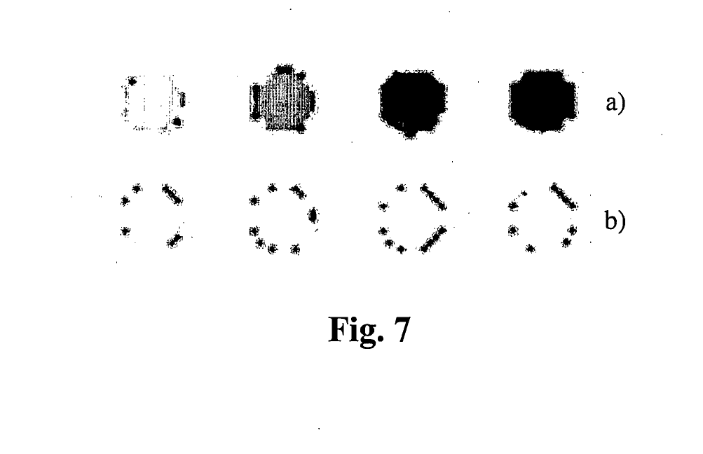

FIG. 7 shows Tᵢ-map of monocytes incubated with 0.1, 0.3, 0.6, and 0.9 mM Gd for 8 hours: a) Gd₂O₃, b) Gd-DTPA.

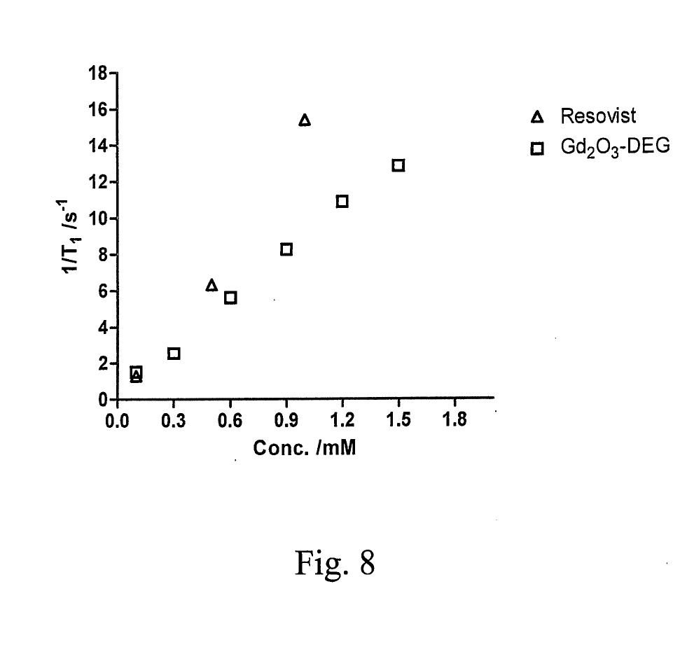

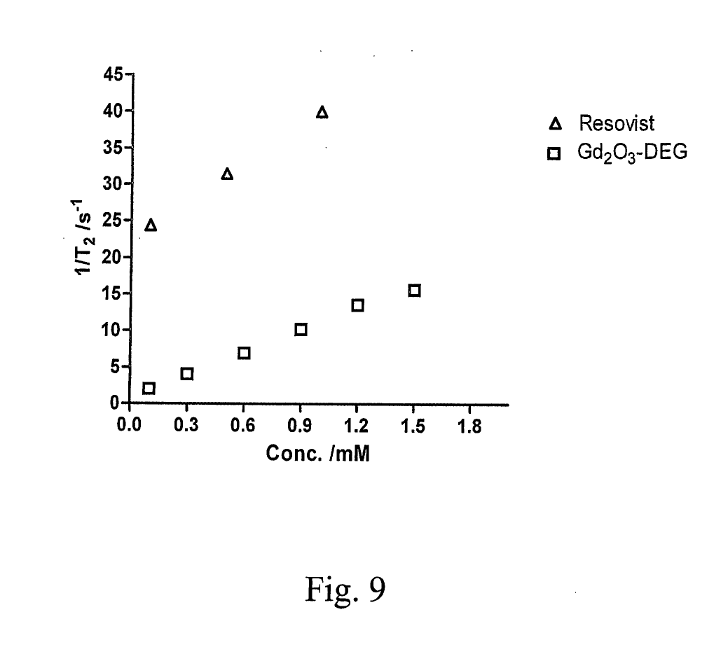

FIGS. 8 and 9 show relaxivity in the form of plots of 1/Tᵢ vs. concentration for Gd₂O₃ nanoparticles according to the present invention and Resovist®.

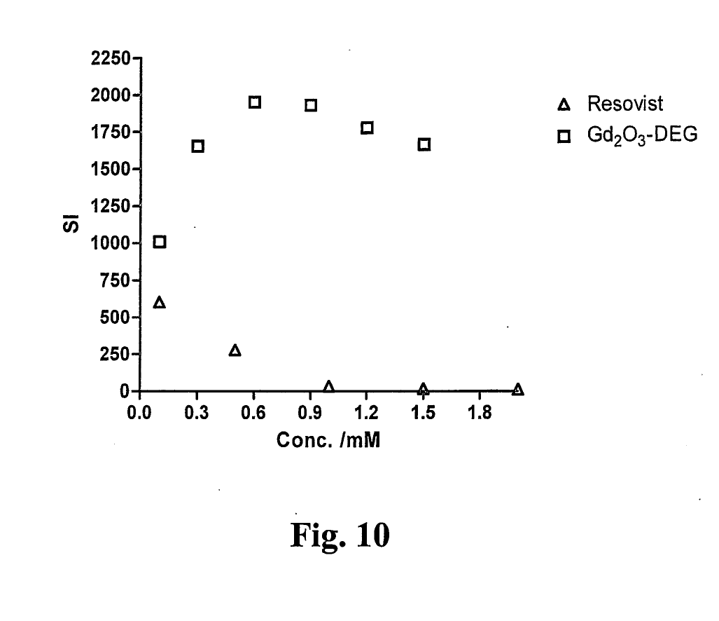

FIG. 10 shows signal intensity from first echo (TE=30 ms, TR=500 ms) in the spin echo sequence used for relaxation time measurements of Gd₂O₃ nanoparticles according to the present invention and Resovist®.

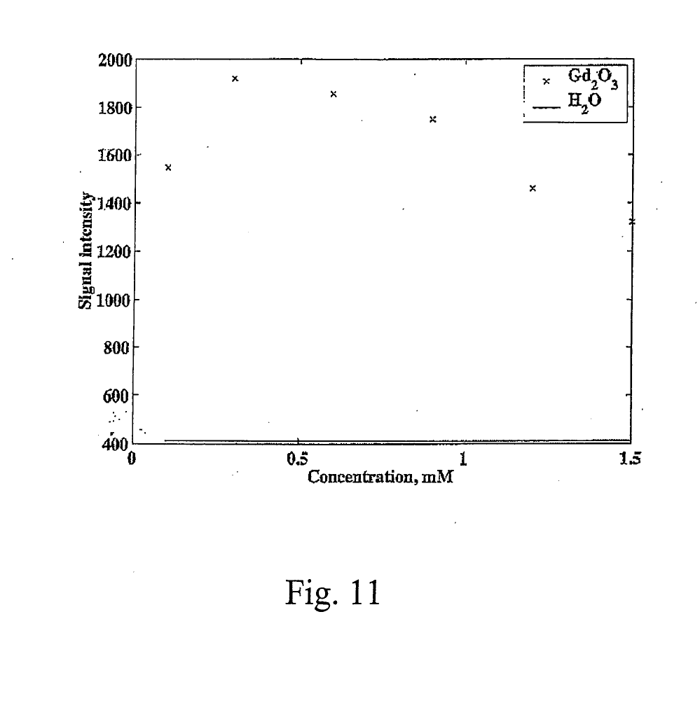

FIG. 11 shows a comparison in signal intensity of water and Gd₂O₃ nanoparticles in concentrations from 0.1 to 1.5 mM Gd.

### EXAMPLE 1

### Synthesis of Gd₂O₃ Nanocrystals Coated with Diethylene Glycol (DEG)

Nanocrystalline gadolinium oxide was synthesized by the polyol method, as described previously in Feldmann C. Polyol-mediated synthesis of nanoscale functional materials. Adv. Funct. Mater. 2003; 13: 101-107; Bazzi R et al., Synthesis and luminescent properties of sub-5-nm lanthanide oxides nanoparticles, Journal of Luminescence. 2003; 102-103: 445-450; and Söderlind, F., et al., Synthesis and characterization of Gd₂O₃ nanocrystals functionalized by organic acids, J. Colloid Interface Sci., 288: 140-148 (2005).

Gd(NO₃)₃6H₂O (2 mmol), solid NaOH (2.5 mmol) and de-ionized water (a few drops) was dissolved in 15 ml diethylene glycol ((HOCH₂CH₂)₂O, DEG) and the mixture is heated to 140° C. When the reactants are completely dissolved, the temperature is raised to 180° C. and held constant for 4 h, yielding a dark yellow colloid. The colloid is diluted with deionized water to adjust the gadolonia concentration to a predetermined value, e.g. 2.5 mM. The concentration was verified by thermogravimetry by heating the sample at 700° C. for 3 h in a carefully cleaned Pt crucible. As corroborated previously by x-ray powder diffraction and transmission electron microscopy, the DEG capped Gd₂O₃ nanocrystals are to large an extent crystalline with sizes in the range of 1 to 15 nm. These crystals were formulated Fractions with narrow distribution: 1-3 nm, 3-6 nm, 6-9 nm, 9-15 nm are obtainable by combined filter/centrifuge separation (VIVASPIN filter obtained from A-filter AB Västra Frölunda, SE).

### EXAMPLE 2

### Synthesis of Gd₂O₃ Nanocrystals Coated with Other Agents, or Alternative Synthesis

Gd(NO₃)₃6H₂O (2 mmol) and NaOH (6 mmol) were dissolved in two separate beakers, each containing 10 ml of DEG. The two solutions were mixed, heated to about 210° C., and held at that temperature for 30 minutes under stirring. To the hot solution oleic acid in DEG (1.6 mmol in 5 ml) was added yielding a brownish syrup. After washing and centrifuging several times in methanol, an off-white powder was collected. Oleic acid was replaced by, respectively, citric acid, 16-hydroxyhexadecanoic acid, 16-aminohexadecanoic acid, or hexadecylamine. In all cases, 1.6 mmol acid/amine in 5 ml DEG were used.

Gd₂O₃ nanocrystals can also be prepared with a rather different method, suitably called a combustion method [W. Zhang, et al., “Optical properties of nanocrystalline Y₂O₃:Eu depending on its odd structure”, J. Colloid and Interface Sc., 262 (2003) 588-593], was performed in the following way. Equal volumes (10 ml) of Gd(NO₃)₃, and the amino acid glycin (each 0.1 M), were mixed in a flask and boiled to near dryness. After one or two minutes of further heating, the brown goo self-ignited and formed a fine, white powder.

### EXAMPLE 3

### Characterisation with X-Ray Photoelectron Spectroscopy (XPS)

In order to confirm that correct Gd₂O₃ nanocrystals were prepared by studying composition and binding energy of the particles, the XPS spectra were recorded on a VG instrument using unmonochromatized Al Kα photons (1486.6 eV) and a CLAM2 analyzer. The power of the X-ray gun was 300 W. The spectra were based upon photoelectrons with a takeoff angle of 30° relative to the normal of the substrate surface. The pressure in the analysis chamber was 3*10⁻¹⁰ mbar and the temperature 297 K during the measurements. The VGX900 data analysis software was used to analyze the peak position. To clean the silicon (SiOₓ) substrates, the surfaces were first washed with a 6:1:1 mixture of MilliQ water: HCl (37%): H₂O₂ (28%) for 5-10 minutes at 80° C. followed by a 5:1:1 mixture of MilliQ water:NH₃ (25%):H₂O₂ (28%) for 5-10 minutes at 80° C. The silicon surfaces were after each washing step carefully rinsed with MilliQ water. Gadolinium oxide nanoparticles capped with diethylene glycol (Gd₂O₃-DEG) were mixed with basic MilliQ water and spin-coated onto freshly cleaned silicon (SiOₓ) substrates at a rate of 2000 rpm and then immediately placed in the XPS instrument.

A wide scan spectrum of the Gd₂O₃-DEG nanoparticles spin-coated on a silicon substrate is presented in FIG. 1a. The most intense photoelectron peaks are found at 1120 eV and 1188 eV. These two peaks originate from Gd (3d_{3/2}) and Gd (3d_{5/2}), respectively. The peak positions are consistent with the oxidation level for Gd₂O₃ [Raiser D, et al.: Study of XPS photoemission of some gadolinium compounds. J Electron Spectrosc. 1991; 57: 91-97]. This is verifying the oxidation level of the sample. The O (1s) peak found at 532 eV, consists of oxygen from three different components, i.e., Gd₂O₃, the capping molecule DEG and the silicon (SiOₓ) substrate. A more detailed analysis on the coordination of the capping molecules to the nanoparticles is in process. The two peaks at 151 eV and 99 eV originate from Si (2s) and Si (2p) as a contribution from the substrate. The film of spin-coated Gd₂O₃-DEG is thin, thus minimizing charging of the sample during the XPS measurements. The prominent peak found at 978 eV, originates from the O (KLL) Auger line.

Samples of Gd₂O₃ nanoparticles made with the combustion method, or capped with oleic acid or citric acid, respectively (preparative methods were in accordance with the procedures earlier disclosed), were also investigated with x-ray photoelectron spectroscopy with same procedure. The Gd (3d) spectrum of oleic acid capped Gd₂O₃ nanocrystals spin-coated onto an SiOₓ substrate is shown in FIG. 1b. The Gd (3d) level consists of a spin orbit split doublet, with the Gd (3d_{5/2}) and Gd (3d_{7/2}) peaks at 1187.7 and 1220.3 eV, respectively. The line shape and peak positions are in good agreement with earlier published data on Gd₂O₃ powder pressed into an In sheet, confirming that the sample consist of Gd₂O₃ (D. Raiser, et al., J. Electron. Spec. 57 (1991) 91-97). The Gd (3d) spectra for citric acid capped particle and particles made with the combustion method were, not surprisingly, identical with that of oleic acid capped particles.

The C (1s) spectrum of oleic acid capped particles shows three different peaks (FIG. 1c). The main peak at 285 eV is assigned to the aliphatic carbons in oleic acid. The peak at about 287 can be assigned to hydroxylcarbons and corresponds to terminating carbons in diethylene glycol. The peak at 289.1 eV corresponds to the carboxyl group in oleic acid. The 0 (1s) spectrum of oleic acid capped particles shows three peaks (FIG. 1d). The peak at 531.1 eV corresponds to the oxygen in the Gd₂O₃ oxide, and the prominent peak at 532.1 eV is, as expected, a contribution from the SiOₓ substrate. The peak at 533 eV originates from the carbonyl carbon in the terminating group of the oleic acid together with C—O—C and C—OH in DEG. The O (1s) spectrum of the citric acid capped particles shows three peaks (FIG. 1e). The ones at 531.2 and 532.3 eV correspond to, respectively, the gadolinia oxygen and the carbonyl group (C═O and/or O—C═O) of citric acid. The third peak at 533.9 eV is related to oxygen in an ester group (C—O—C═O). Ester formation is likely to occur during the synthesis since it involves an alcohol and a carboxylic acid. The O (1s) spectrum of the sample from the combustion synthesis also shows three peaks (FIG. 1f). As above (FIGS. 1d and 1e), the peak at 531.2 eV corresponds to the gadolinia oxygen. The dominating peak at 532.3, and the smaller one at 535.6 eV, are interesting. The former can be assigned to carbonyl oxygen (C═O and/or O—C═O), and the latter to oxygen with nitrogen as nearest neighbour. The sources for these peaks are either unreacted reactants (glycine, gadolinium nitrate) and/or carbonyl and nitrogen containing reaction products.

### EXAMPLE 4

### Characterization with Transmission Electron Microscopy (TEM)

The TEM studies were carried out with a Philips CM20 electron microscope, operated at 200 kV. The size of the Gd₂O₃ nanoparticles prepared via the DEG route were about 5 nm, as seen in the HREM micrograph in FIG. 3. Although the contrast is poor, the (222) planes (d≈3.1 Å) are visible. A HREM micrograph of a nanocrystal obtained in the synthesis with oleic acid, approximately 15 nm in diameter, is shown in FIG. 4. There is no contrast from the capping layer (if it is there), but unbroken (222) planes running throughout the crystal can be seen. A TEM image of nanocrystals obtained in the combustion synthesis is shown FIG. 5. An aggregate of at least three nanocrystals are visible and the size is about 10 nm, or less. The results from TEM uniformly showed that crystalline nanoparticles were obtained.

### EXAMPLE 5

### Sample Preparation

Samples of Gd₂O₃-DEG from Example 1 (with an average particle size of about 5 nm and a particle size range from about 1 to 15 nm) and Gd-DTPA (Magnevist®) were prepared in 10 mm NMR test tubes with H₂O at 9 different Gd concentrations from 0.1-2.5 μM. At measurement the test tubes were immersed in a bowl with saline at 22-23° C., which was the temperature of the scanner room.

### EXAMPLE 6

### Relaxation Time Measurements and Magnetic Resonance Imaging

T₁ and T₂ relaxation times were measured with a 1.5 T Philips Achieva whole body scanner using the head coil. A 2D mixed multi-echo SE interleaved with multi-echo IR sequence was used for the measurements [kleef_mrm_{—}1987]. Imaging time parameters were varied to minimise the standard deviations in relaxation time calculations: TE=30 ms, TR (SE)=500 ms, TI=150 ms, and TR (IR)=1150 ms (set 1); TE=50 ms, TR (SE)=760 ms, TI=370 ms, and TR (IR)=2290 ms (set 2). Other MR parameters were: FOV=23 cm, slice thickness=7 mm, number of echoes=4.

A substantial increase in proton relaxivity for the gadolinium nanoparticles in H₂O compared to Gd-DTPA was achieved. Table 1 shows that the relaxivities of Gd₂O₃ were almost twice the values for Gd-DTPA: r₁(Gd₂O₃)/r₁(Gd-DTPA)=1.89, r₂(Gd₂O₃)/r₂(Gd-DTPA)=1.94. The plots of 1/Tᵢ vs gadolinium concentration in FIGS. 5a and 5b show a linear relationship with a good fit (r₁, r₂>0.99, Tab. 1), according to Eq. 2.

TABLE 1
Relaxivity constants (r₂, r₂) and goodness of
fit (r₁, r₂) for Gd-DTPA (Magnevist ®) and Gd₂O₃-DEG.

| | r₁ | | r₂ | |
|---|---|---|---|---|
| | (mM⁻¹ s⁻¹) | r₁ | (mM⁻¹ s⁻¹) | r₂ |
| Gd-DTPA | 4.86 ± 0.08 | 0.9983 | 5.53 ± 0.14 | 0.9975 |
| Gd₂O₃-DEG | 9.19 ± 0.10 | 0.9984 | 10.74 ± 0.27 | 0.9957 |

Analysis of the signal intensity showed higher signal intensity at lower concentrations of Gd₂O₃ samples compared to Gd-DTPA, using data from the first echo in the spin echo part of the sequence used for relaxation time measurements, TE=30 ms, TR=500 ms (FIG. 6). At higher concentrations (>0.9 mM) the strong T₂ effect attenuated the signal for the nanoparticle samples. That is, the Gd₂O₃ sample reached the signal intensity peak at lower concentrations (0.6 mM) compared to the Gd-DTPA signal intensity that peaked at approximately 1.2 mM in this sequence.

The analysis showed a considerable increase in relaxivity for Gd₂O₃ in H₂O compared to Gd-DTPA. Another interesting feature of these experiments was the marked Tᵢ reducing effect and consequential signal increase seen at low concentrations. The concentration range below 0.6 mM in plasma is the one most relevant for clinical applications. At a dose of Magnevist 0.1 mmol/kg (as recommended by the manufacturer), the detected plasma concentration of Gd is 0.6 mM at 3 minutes after injection and 0.24 mM at 60 minutes after injection (Data provided by the Medical Product Agency of Sweden, FASS).

The signal intensity for Gd₂O₃ in the spin echo sequence illustrated in FIG. 6 both raised and dropped more rapidly than the Gd-DTPA signal. The steep signal increase at low concentration (<0.6 mM) can be explained by the high T₁ relaxivity. However, at higher concentration the T₂ lowering effect was more pronounced for the Gd₂O₃ particles. The faster signal drop can be caused by susceptibility effects due to magnetic field inhomogeneity at particle sites.

In addition, samples of Gd₂O₃-DEG and Resovist® were prepared and tested under the same conditions as above. There were 6 different Gd and Fe concentrations between 0.1 and 1.5 mM. Resovist® is based on ferrocarbotran colloidal sol of superparamagnetic iron oxide nanoparticles (SPIO). The particles have a hydrodynamic diameter of 60 nm on an iron core of 4 nm. The relaxivities and signal intensities are shown in FIGS. 8, 9 and 10. These results demonstrate that Resovist® has a higher T₁ and T₂ relaxivity compared with Gd₂O₃-DEG. When comparing the curves, it is obvious that Resovist® has a significantly higher T₂ relaxivity. This means that Resovist provides a negative contrast compared to Gd₂O₃-DEG, which provides a positive contrast (c.f. the signal intensity curves in FIG. 10). Accordingly, Gd₂O₃-DEG particles enable a contrast agent with complementary properties to those based on SPIO.

For the comparison of Gd₂O₃ signal intensity with that of water, as shown in FIG. 11, the signal intensity was achieved with first echo in the spin sequence (TE=30 ms, TR=500 ms) relaxation time measurements. The test tubes were immersed in saline allowing for simultaneous measurements of signal intensity in water and Gd₂O₃ samples.

### EXAMPLE 7

### Monocyte Studies

For monocyte experiments, THP-1 cells were cultured in RPMI 1640 medium with 10% fetal calf serum (GIBCO, Invitrogen, Carlsbad, Calif., USA) with additions of L-glutamate and penicillin/streptomycin solution (Invitrogen). Cells were counted and found 97% viable. Cells were treated with Gd₂O₃-DEG or Gd-DTPA in concentrations 0.1, 0.3, 0.6, and 0.9 mM. Cells of one well were left untreated. A control series was prepared of cell culture medium only with the different concentrations of Gd₂O₃-DEG particles. Two plates of 24 wells each were prepared as described above; one was incubated with the Gd₂O₃-DEG and Gd-DTPA for 2 h and the other one for 8 h at 37° C. After incubation cells were transferred to Falcon tubes and washed twice with medium and centrifugated for 8 minutes at 1100 rpm. The monocyte experiments showed that the nanoparticles after the washing procedure, were either attached to cell surfaces or internalized by the cells. Gd-DTPA was not present in the cell suspension after the washing. Both T₁ and T₂ relaxation times decreased at higher Gd₂O₃ concentrations and also at the longer incubation time (data not shown). A T₁ map resulting from the 8 hour incubation is shown in FIG. 7.

Magnevist (Gd-DTPA) is manufactured to remain in the extracellular space. In FIG. 4b it is seen that Gd-DTPA was effectively washed out from the sample. On the contrary, Gd₂O₃ remained in cell cultures after washing. (FIG. 4a). It has been shown that certain cell types, such as macrophages can internalize small particles through phagocytosis [Weissleder R, et al.: Magnetically labelled cells can be detected by MR imaging, J Magn Res Imag. 1997; 7: 258-263]. Earlier studies on THP-1 cells incubated with iron oxide nanoparticles show a linear relation between cell uptake and dose/incubation time [Bowen C V, et al.: Application of the static dephasing regime theory to superparamagnetic iron-oxide loaded cells, Magn Res Med. 2002; 48: 52-61]. These earlier results indicate that also in the present experiments Gd-particles may have probably were taken up by the cells.

1. Superparamagnetic nanoscale particles, comprising gadolinium oxide having average sizes between about 0.5 to 50 nm.

2. Particles according to claim 1, having a biocompatible and/or biospecific coating.

3. Particles according to claim 2, having a coating comprising diethylene glycol (DEG) and/or citric acid.

4. Particles according to claim 2, having a coating comprising folic acid.

5. Particles according to claim 4, wherein the coating comprises polyethylene glycol linked to folic acid.

6. Particles according to claim 1 with an average size of about 5 nm having a coating comprising diethylene glycol (DEG).

7. A composition comprising the particles according to claim 1.

8. A composition according to claim 7, having a gadolinium concentration of 0.01 to 500 mM.

9. A composition according to claim 7 adapted for administration to a body site.

10. A composition according to claim 9 comprising a parenterally administerable vehicle.

11. A composition according to claim 7, wherein the composition has a capacity to reduce relaxation times T₁ and/or T₂ of neighbouring hydrogen nuclei in a proton rich environment below values of T₁ and/or T₂ obtained by a composition of an ionic complex of gadolinium.

12. A composition according to claim 7 having a higher signal intensity than obtained by nanoscale iron oxide particles in the concentration range of 0.1 mM to 1.5 mM.

13. A contrast agent comprising the composition of claim 7.

14. A contrast agent according to claim 13, having at least 500% better signal intensity than water.

15. A method of performing MRI (magnetic resonance imaging) comprising administering a contrast agent according to claim 13 for studying molecular interactions or cellular processes.

16. (canceled)

17. A composition according to claim 7, having a gadolinium concentration of 0.01 to 2.5 mM.

18. A composition according to claim 8, adapted for administration to a body site.

19. A contrast agent according to claim 13, having at least 700% better signal intensity than water.

20. A method of performing MRI (magnetic resonance imaging) comprising administering a contrast agent according to claim 14 for studying molecular interactions or cellular processes.

21. A method of performing MRI (magnetic resonance imaging) comprising administering a contrast agent according to claim 19 for studying molecular interactions or cellular processes.
